(19) United States
(12) Patent Application Publication
(10) Pub. No.: US 2016/0326555 A1
(43) Pub. Date: Nov. 10, 2016

(54) ENZYMATIC METHOD

(71) Applicant: EVONIK DEGUSSA GMBH, Essen (DE)

(72) Inventors: Philip ENGEL, Essen (DE); Thomas HAAS, Muenster (DE); Wolfgang KROUTIL, Graz (AT); Johann H. SATTLER, Bad Aussee (AT)

(73) Assignee: EVONIK DEGUSSA GmbH, Essen (DE)

(21) Appl. No.: 15/111,640

(22) PCT Filed: Dec. 18, 2014
(86) PCT No.: PCT/EP14/78465

§ 371 Date: Jul. 14, 2016

(30) Foreign Application Priority Data
Jan. 14, 2014 (DE) 10 2014 200 497.7

(51) Int. Cl. C12P 7/62

(52) U.S. Cl. CPC C12P 7/62

(57) ABSTRACT

The present invention relates to a method comprising the method steps A) Providing at least one compound of the general formula I) where X=divalent organic radical comprising 1 to 19 carbon atoms B) Contacting the compound of the general formula I) with an enzyme E₁ selected from the group esterases, lipases and lactonases, characterized in that method step B) is carried out in the presence of at least one aliphatic alcohol comprising 1 to 6 carbon atoms.

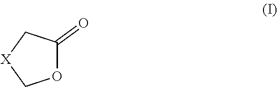

### FIELD OF THE INVENTION

The present invention relates to a method comprising the method steps
A) Providing at least one compound of the general formula I)

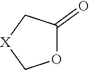

where X=divalent organic radical comprising 1 to 19 carbon atoms
B) Contacting the compound of the general formula I) with an enzyme E₁ selected from the group
Esterases, lipases and lactonases,
characterized in that method step B) is carried out in the presence of at least one aliphatic alcohol comprising 1 to 6 carbon atoms.

### PRIOR ART

Macromolecules, 1995, 28, 73-78, Macromolecules, 2004, 37, 2450-2453 and Macromolecules 2006, 39, 7967-7972 disclose enzymatic methods for hydrolysis of lactones with lipases, esterases and lactonases in non-aqueous media. Under the conditions described, the resultant products polymerize, in such a manner that further modification at the ω-position, for example in the form of an amination, is not possible.

The object of the invention was to provide an enzymatically catalyzed method for ring opening of lactones, which makes possible further modification at the ω-position.

### DESCRIPTION OF THE INVENTION

Surprisingly, it has been found that the method described hereinafter is able to solve the problem posed by the invention.

The present invention therefore relates to a method comprising the method steps
A) Providing at least one compound of the general formula I)

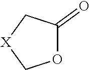

where X=divalent organic radical comprising 1 to 19 carbon atoms
B) Contacting the compound of the general formula I) with an enzyme E₁ selected from the group
esterases of EC 3.1, lipases of EC 3.1.1 and lactonases of EC 3.1.1,
characterized in that method step B) is carried out in the presence of at least one aliphatic alcohol comprising 1 to 6 carbon atoms.

One advantage of the present invention is that the conversion rate of the method is very high. Therefore, the method can be carried out in a reduced time.

A further advantage of the present invention is that the yields are increased.

A further advantage of the present invention is that the products can be purified in a simplified manner.

A further advantage is that the reaction proceeds highly selectively and no ring opening takes place without conversion to the ester.

The accession numbers listed in connection with the present invention correspond to the protein bank database entries of the NCBI with a date of 01.10.2013; generally, in the present case, the version number of the entry is identified by “.number” such as, for example, “1”.

Where documents are cited in the context of the present description, it is intended that their content fully form part of the disclosure content of the present invention.

Unless stated otherwise, all of the stated percentages (%) are percent by mass.

The method according to the invention is suitable, in particular, for producing ω-hydroxycarboxylic esters, wherein the compound of the general formula I) is esterified, with ring opening, with the aliphatic alcohol.

In a preferred embodiment of the method according to the invention, X is selected from optionally substituted alkylene groups, preferably —(CH₂)—, —(CH₂)₂—, —(CH₂)₃—, —(CH₂)₄—, —(CH₂)₅—, —(CH₂)₆— and —(CH₂)₇—, particularly preferably —(CH₂)₂—, —(CH₂)₃— and —(CH₂)₄—.

The aliphatic alcohol is preferably selected from methanol, ethanol, propanol, isopropanol and butanol, wherein methanol is particularly preferred.

The aliphatic alcohol in method step B) in particular is used at a concentration of 5% by weight to 60% by weight, preferably from 10% by weight to 40% by weight, particularly preferably from 12% by weight to 30% by weight, wherein the percentages by weight relate to the total reaction batch.

A method preferred according to the invention is characterized in that method step B) is carried out in an aqueous environment. The expression “aqueous environment” is preferably taken to mean that water is used at a concentration of 3% by weight to 95% by weight, preferably from 5% by weight to 90% by weight, particularly preferably from 10% by weight to 85% by weight, wherein the percentages by weight relate to the total reaction batch.

Method step B) of the method according to the invention is preferably carried out in a temperature range from 5° C. to 80° C., preferably from 15° C. to 60° C., particularly preferably from 25° C. to 40° C.

Method step B) of the method according to the invention is preferably carried out in a pH range from 3 to 11, preferably from 5 to 9, particularly preferably from 6.5 to 8.

The “pH” in connection with the present invention is defined as the value which is measured using a calibrated pH electrode as specified in ISO 4319 (1977) for a corresponding composition at 25° C. after stirring for 5 minutes.

Preferably, according to the invention, the enzyme E₁ is selected from esterases in which the access tunnel which the substrate must pass along in order to arrive at the active centre is hydrophobic and it is characterized by a hydrophobicity index of 0.1 to 1.8. The hydrophobicity index is determined as described in Journal of Cheminformatics 2013, 5:39 doi:10.1186/1758-2946-5-39 and Protein Eng. (1992) 5 (5): 373-375. doi: 10.1093/protein/5.5.373.

According to the invention, preferably, the enzyme E₁ is selected from the group

XP_003364701.1 (predicted equus caballus carboxylesterase isoform X2),

XP_005608328.1 (predicted equus caballus carboxylesterase isoform X3),

NP_388425.1 (Esterase 008 SD Bacillus subtilis),

Pig Liver Esterase 03 (commercially available from Enzymicals as ECS-PLE03),

Pig Liver Esterase 06 (commercially available from Enzymicals as ECS-PLE06), and also

CAO81735.1 (Alternative Pig liver esterase),

GI:576155 (Horse pancreatic lipase Chain A: 1HPL_A),

GI:576156 (Horse pancreatic lipase Chain B: 1HPL_B) and

AAC12774.1 (Esterase from Bacillus subtilis pdb 1JKM brefeldin A esterase),

and also proteins having a polypeptide sequence in which up to 60%, preferably up to 25%, particularly preferably up to 15%, in particular up to 10, 9, 8, 7, 6, 5, 4, 3, 2, 1% of the amino acid residues are modified compared with the abovementioned reference sequences by deletion, insertion, substitution or a combination thereof, and which still possess at least 50%, preferably 65%, particularly preferably 80%, in particular more than 90%, of the activity of the protein having the corresponding abovementioned reference sequence, wherein 100% activity of the reference protein is taken to mean the amount of substance of ε-caprolactone, based on the amount of reference enzyme used that is reacted with methanol to give the corresponding ester per unit time.

A method for determining the activity is described in Example 1.

It is preferred according to the invention if, in addition to the enzyme E₁ in method step B), at least one enzyme selected from E₂, E₃ and E₄ is used, wherein

the enzyme E₂ catalyses the reaction of ω-hydroxycarboxylic esters to give the corresponding ω-oxocarboxylic esters,

the enzyme E₃ catalyses the reaction of ω-oxocarboxylic esters to give the corresponding ω-aminocarboxylic esters and

the enzyme E₄ catalyses the reaction of ω-oxocarboxylic esters to give the corresponding ω-aminocarboxylic acids.

The enzymes E₁, E₂, E₃ and E₄ can therefore be used as enzyme combinations selected from E₁, E₁E₂, E₁E₃, E₁E₄, E₁E₂E₃, E₁E₃E₄, E₁E₂E₄ and E₁E₂E₃E₄.

Depending on the choice of the enzyme combination, the method can be used for producing ω-hydroxycarboxylic esters (e.g. E₁), for producing ω-oxocarboxylic esters (e.g. E₁E₂), for producing ω-aminocarboxylic esters (e.g. E₁E₂E₃) and/or for producing ω-aminocarboxylic acids (e.g. E₁E₂E₃E₄).

Methods according to the invention in which in method step B) enzyme combinations comprising E₁E₂E₃E₄ are used preferably have the aliphatic alcohol at a concentration of 12% by weight to 22% by weight, wherein the percentages by weight relate to the total reaction batch and particularly preferably, this concentration is combined with a temperature range in method step B) from 25° C. to 40° C.

Methods preferred according to the invention are characterized in that E₂ is selected from alkane monooxygenases, alcohol dehydrogenases and alcohol oxidases.

Suitable enzymes E₂ are described, for example, as “enzyme E_{1I}” in EP2222844 and as “NAD(P)+-dependent alcohol dehydrogenases” in WO2013011018.

Preferably according to the invention, the enzyme E₂ is selected from the group P42328.1 (ADH-hT), NP_415995.4 (primary ADH from Escherichia coli), ACB78191.1 (Ralstonia eutropha), YP_795183.1 (Lactobacillus brevis), ACF95832.1 (Lactobacillus kefiri), ACB78182.1 (Paracoccus pantotrophus), EU427523.1 (Sphingobium yanoikuyae), AY123972.1 (Arthrobacter sp. BP2),

and also

proteins having a polypeptide sequence in which up to 60%, preferably up to 25%, particularly preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof, and which still possess at least 50%, preferably 65%, particularly preferably 80%, in particular more than 90%, of the activity of the protein having the corresponding abovementioned reference sequence, wherein 100% activity of the reference protein is taken to mean the amount of substance of ω-hydroxyhexanoic acid methyl ester based on the amount of reference enzyme used that is reacted to give the corresponding ω-oxocarboxylic ester per unit time.

A method for determining the activity is described hereinafter. The activity is determined by photometric measurement of the change in absorption from NAD⁺ to NADH (0.5 mM) at 340 nm in the presence of a substrate (6-hydroxyhexanoic acid methyl ester, 50 mM) and of the suitable enzyme from the group E₂.

Methods preferred according to the invention are characterized in that E₃ is selected from ω-transaminases.

Suitable enzymes E₃ are described, for example, as “enzyme E_{1II},” in EP2222844, as “polypeptide having transaminase activity” in EP2557176 and as “transaminases” in WO2013011018.

Preferably according to the invention, the enzyme E₃ is selected from the group NP_901695.1 (Chromobacterium violaceum), YP_917746.1 (Paracoccus denitrificans), and also BAK39753.1 (Arthrobacter sp.), and also the abovementioned suitable enzymes E₃, and also proteins having a polypeptide sequence in which up to 60%, preferably up to 25%, particularly preferably up to 15%, in particular up to 10, 9, 8, 7, 6, 5, 4, 3, 2, 1% of the amino acid residues are amended in comparison with the abovementioned reference sequences by deletion, insertion, substitution or a combination thereof and which still possess at least 50%, preferably 65%, particularly preferably 80%, in particular more than 90%, of the activity of the protein having the corresponding abovementioned reference sequence, wherein 100% activity of the reference protein is taken to mean the amount of substance of ω-oxohexanoic acid methyl ester based on the amount of reference enzyme used reacted to give the corresponding ω-aminocarboxylic ester per unit time.

A method for determining the activity is described hereinafter. The activity of the lyophilised cells (20 mg) is determined using a time-degree of conversion curve, wherein the conversion of the substrate (6-oxohexanoic acid methyl ester, 50 mM) to the amine is determined at various time points (0.5 h, 1 h, 2 h, 4 h, 6 h, 12 h, 20 h). Previously, the rehydration of the cells is performed by shaking (120 rpm, 30 min) with addition of PLP (pyridoxal phosphate) (0.4 μmol). The NAD⁺ is recycled to NADH (0.5 mM) using a GDH (1 U) and glucose (100 mM). The linear part of the curve is used to calculate the activity.

Preferably according to the invention, the enzyme E₄ is selected from the group

XP_003364701.1 (predicted equus caballus carboxylesterase isoform X2),

XP_005608328.1 (predicted equus caballus carboxylesterase isoform X3),

NP_388425.1 (Esterase 008 SD Bacillus subtilis),

Pig Liver Esterase 03 (commercially available from Enzymicals as ECS-PLE03),

Pig Liver Esterase 06 (commercially available from Enzymicals as ECS-PLE06), and also

CAO81735.1 (Alternative Pig liver esterase),

GI:576155 (Horse pancreatic lipase Chain A: 1HPL_A),

GI:576156 (Horse pancreatic lipase Chain B: 1HPL_B) and

AAC12774.1 (Esterase from Bacillus subtilis pdb 1JKM brefeldin A esterase), and also proteins having a polypeptide sequence in which up to 60%, preferably up to 25%, particularly preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still possess at least 50%, preferably 65%, particularly preferably 80%, in particular more than 90%, of the activity of the protein having the corresponding abovementioned reference sequence, wherein 100% activity of the reference protein is taken to mean the amount of substance of ω-aminohexanoic acid methyl ester based on the amount of reference enzyme used reacted to give the corresponding ω-aminocarboxylic acid per unit time.

A method for determining the activity is described in Example 1.

It is possible, and preferred according to the invention, that the enzyme E₁ respectively used can perform the enzymatic activity of the enzyme E₄; such enzymes are termed above preferred enzymes E₁.

It is preferred according to the invention if, apart from the possible enzyme combinations formed from E₁, E₂, E₃ and E₄ in method step B), in addition at least one enzyme E₅ selected from alanine dehydrogenases is used.

It is a particular strength of the present invention that this configuration permits a reduction-equivalent-neutral reaction procedure, i.e. the reaction proceeds without supply or removal of electrons in the form of reduction equivalents, since the NAD(P)H generated by the alcohol dehydrogenase in the course of the alcohol oxidation is consumed in the generation of alanine with consumption of an inorganic nitrogen donor, preferably ammonia or an ammonia source. The alanine in turn can be used by the transaminase that is present.

In a preferred embodiment, the term “alanine dehydrogenase”, as used herein, is understood as meaning an enzyme which catalyses the conversion of L-alanine with consumption of water and NAD(P)⁺ to pyruvate, ammonia and NAD(P)H. Preferably, the alanine dehydrogenase is an intracellular alanine dehydrogenase.

Preferably, according to the invention, the enzyme E₅ is selected from the group:

Alanine dehydrogenase from Bacillus subtilis (NP_391071.1)

Rhizobium leguminosarum (YP_0029754337.1)

Bacillus megaterium (YP_003565624.1)

Rhodobacter capsulatus (ADE84249.1)), and also

proteins having a polypeptide sequence in which up to 60%, preferably up to 25%, particularly preferably up to 15%, in particular up to 10, 9, 8, 7, 6, 5, 4, 3, 2, 1% of the amino acid residues in comparison with the abovementioned reference sequences are modified by deletion, insertion, substitution or a combination thereof, and which still possess at least 50%, preferably 65%, particularly preferably 80%, in particular more than 90%, of the activity of the protein having the corresponding abovementioned reference sequence.

Enzyme combinations preferably used in this connection are selected from E₁E₂E₅, E₁E₂E₃E₅, E₁E₂E₄E₅, and E₁E₂E₃E₄E₅.

It is preferred according to the invention if, in the method according to the invention, the respective enzymes of the possible enzyme combinations are used in the form of genetically modified cells which have an increased activity of the abovementioned enzymes in comparison with the wild type thereof.

The examples listed below illustrate the present invention by way of example, without any intention of restricting the invention, the scope of application of which is apparent from the entirety of the description and the claims, to the embodiments specified in the examples.

### EXAMPLES

### Example 1

### Esterases for Ring Opening of ε-Caprolactone in the Presence of Methanol

Methanol (100 μl, 10% v/v) was added to buffer (870 μl, 100 mM Na₂KPO₄, pH 8.5), together with enzyme (6 mg, rehydrated for 20 min in 30 μl buffer).

The reaction was started by adding 6 μl (50 μM) of ε-caprolactone. After one hour the reaction was stopped by extraction with 600 μl of ethyl acetate. After phase separation, the aqueous phase was again extracted with 600 μl of ethyl acetate and the two collected organic phases were then combined, dried with Na₂SO₄ and analysed by GC-MS.

As enzyme, the commercial preparations

Bacillus subtilis “Esterase 008-SD” from Codexis, (NP_388425.1) and

“horse liver esterase” were used.

In all cases, a conversion to 6-hydroxyhexanoic acid methyl ester was observed.

The “horse liver esterase” used is a mixture of two isoenymes XP_003364701.1 and

XP_005608328.1 (predicted horse liver esterase), cf. in this context Comp. Biochem. Physiol. D: Genomics Proteomics 2009, 4, 54-65.

### Example 2

### Combination of Esterase with Alcohol Dehydrogenase and Transaminase

L-alanine (250 mM) and NH₄Cl (150 mM) were charged in Eppendorf tubes (2 ml). The pH was adjusted to 8.5 using 6 M NaOH aq. NAD+ (0.5 mg, 0.6 μmol, in 50 μl of H₂O), the transaminase ω-factor PLP (0.1 mg, 0.4 μmol, in 50 μl of H₂O) and methanol (100 μl, 10% v/v) were added together with 300 μl of H₂O.

Thereafter, the respective enzymes were added in accordance with the table hereinafter. For the ADHs (E₂), in the case of ADH-hT (P42328.1) 100 μl of purified enzyme (0.4 U) were added, alternatively, the 6-hydroxyhexanoate dehydrogenase (chnD) from Arthrobacter sp. BP2 (AY123972.1) was used. Of the transaminase (E₃), in the case of transaminase, 20 mg of lyophilised cells were added. The two transaminases used are transaminase from Ralstonia eutropha (RalEu) (YP_917746.1) and transaminase from Paracoccus denitrificans (TA_ParDen) (YP917746.1). Of the esterase (E₁) from Bacillus subtilis (008) (NP_388425.1) and from horse liver (HL) (XP_003364701.1 and XP_005608328.1), 15 μl (10 U according to the manufacturer) of the commercially available enzyme were used. The experiment was carried out at 30° C. and 120 rpm for 18 h at pH 7.8-8.5 (adjusted using 6 M NaOH).

The reaction was started by adding ε-caprolactone (6 μl, 50 mM). After 5 min, the pH was increased to 7.5-8.5 by adding NaOH (6 M, 12 μl).

The results of the reaction with the described enzyme cascade are summarized in the following table.

| | | | | 6-aminohexanoic acid^{[a]} |
|---|---|---|---|---|
| No. | E₁ | E₂ | E₃ | [%] |
| 1a | 008 | chnD | RalEu | ~7 |
| 1b | HL | | | 2 |
| 2a | 008 | hT | | 2 |
| 2b | HL | | | 2 |
| 3a | 008 | hT | TA_ParDen | ~40 |
| 3b | HL | | | ~75 |

^{[a]}The analysis does not differentiate between 6-aminohexanoic acid and its esters.

### Example 3

### Effect of Methanol Concentration and Temperature

Alanine (250 mM) was charged in an Eppendorf Tube (2 ml). Buffer was added (Na₂HPO₄/KH₂PO₄ 300 mM, pH 8.0, 300 μl). NH₄Cl was added (50 μl, 267.1 mg dissolved in 1.25 ml of H₂O and supplemented with 20 μl of 6 M NaOH aq). NAD+ (0.3 mg, dissolved in 50 μl of buffer) and PLP (0.05 mg dissolved in 50 μl of buffer; 1 mg/ml of stock solution) were added. The mixtures were shaken up to homogeneous solution at 37° C., 120 rpm, for approximately 10 min.

The effect of the ω-solvent methanol was studied on the conversion of ε-caprolactone to 6-aminohexanoic acid. Of the transaminase (TA_ParDen), 20 mg of lyophilised cells were rehydrated for 15 min. Of the ADH-hT, 100 μl, equivalent to an activity of 0.4 U on the substrate 6-hydroxyhexanoic acid were used. Of the AlaDH from Bacillus subtilis (NP_388425.1), 10 μl having an activity of 0.22 U for alanine as substrate were used. Of the esterase PLE03, 2 mg, equivalent to an activity of 0.66 U, were added.

In addition, NH₄Cl (8 mg, 150 mM), Alanine (22.3 mg, 250 mM), PLP (0.05 mg, 0.18 mM), NAD⁺ (0.5 mg, 0.75 mM) were added. The experimental conditions were 30° C., 120 rpm, 18 h, pH 7.8-8.5 (adjusted using 6M NaOH). The experiment was started by adding the reactant (6 μL OF ε-caprolactone (50 mM))

The table below shows that with increasing MeOH concentration, formation of 6-aminohexanoic acid is favoured.

| MeOH | | | 6- |
|---|---|---|---|
| v/v | ε-capro- | 6-Hydroxyhexanoic | Aminohexanoic |
| [%] | lactone | acid [%] | acid [%] |
| 2 | 0 | 78 | 22 |
| 3 | 0 | 65 | 35 |
| 5 | 0 | 57 | 43 |
| 10 | 0 | 49 | 51 |

To study the effect of the pH, two different buffer systems were tested similarly to the abovementioned example. The results are summarized in the following table:

| | | | | | 6- | 6- |
|---|---|---|---|---|---|---|
| | | | | | Hydroxyhex- | Aminohex- |
| | MeOH | | | ε- | anoic | anoic |
| | v/v | | buffer | Capro- | acid + ester | acid + ester |
| Entry | [%] | pH | (mM) | lactone | [%] | [%] |
| 1 | 2 | 6.8-7.4 | PIPES | 0 | 87 | 13 |
| | | | (120) | | | |
| 2 | 5 | 6.8-7.4 | PIPES | 0 | 85 | 15 |
| | | | (120) | | | |
| 3 | 10 | 6.8-7.4 | PIPES | 0 | 78 | 22 |
| | | | (120) | | | |
| 4 | 15 | 6.8-7.4 | PIPES | 0 | 73 | 27 |
| | | | (120) | | | |
| 5 | 2 | 6.8-7.4 | PO₄ | 0 | 86 | 14 |
| | | | (120) | | | |
| 6 | 5 | 6.8-7.4 | PO₄ | 0 | 80 | 20 |
| | | | (120) | | | |
| 7 | 10 | 6.8-7.4 | PO₄ | 0 | 71 | 29 |
| | | | (120) | | | |
| 8 | 15 | 6.8-7.4 | PO₄ | 0 | 65 | 35 |
| | | | (120) | | | |

In addition, the effect of temperature was likewise studied using the experimental setup described:

| | | | | | 6- | 6- |
|---|---|---|---|---|---|---|
| | | | | ε- | hydroxyhex- | Aminohex- |
| | MeOH | | | capro- | anoic | anoic |
| | v/v | | Temp. | lactone | acid + ester | acid + ester |
| Entry | [%] | pH | [° C.] | [%] | [%] | [%] |
| 1 | 20 | 6.8-7.4 | 30 | 0 | 76 | 24 |
| 2 | 25 | 6.8-7.4 | 30 | 16 | 74 | 10 |
| 3 | 10 | 6.8-7.4 | 37 | 0 | 80 | 20 |
| 4 | 15 | 6.8-7.4 | 37 | 2 | 64 | 34 |
| 5 | 20 | 6.8-7.4 | 37 | 13 | 76 | 11 |
| 6 | 20 | 8.7-9.0 | 30 | 19 | 76 | 5 |

Analogous results using Bacillus subtilis 008 SD as E₁:

| | MeOH | | | 6- | 6- |
|---|---|---|---|---|---|
| | v/v | Temp. | ε-Capro- | Hydroxyhexanoic | Aminohexanoic |
| Entry | [%] | [° C.] | lactone | acid [%] | acid [%] |
| 1 | 15 | 30 | 0 | 60 | 40 |
| 2 | 20 | 30 | 0 | 75 | 25 |
| 3 | 30 | 30 | 27 | 64 | 9 |
| 4 | 35 | 30 | 33 | 67 | 0 |
| 5 | 15 | 37 | 0 | 63 | 37 |
| 6 | 20 | 37 | 2 | 96 | 2 |
| 7 | 30 | 37 | 30 | 70 | 0 |

1. Method comprising the method steps
A) Providing at least one compound of the general formula 1) 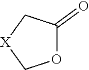
where X=divalent organic radical comprising. 1 to 19 carbon atoms
B) Contacting the compound of the general formula I) with an enzyme E₁ selected from the group esterases of EC 3.1, lipases of EC 3.1.1 and lactonases of EC 3.1.1,
characterized in that method step B) is carried out in the presence of at least one aliphatic alcohol comprising 1 to 6 carbon atoms.

2. Method according to claim 1, characterized in that X is selected from optionally substituted alkylene groups, preferably —(CH₂)—, —(CH₂)₂—, —(CH₂)₃—, —(CH₂)₄—, —(CH₂)₅—, —(CH₂)₆— and —(CH₂)₇—, particularly preferably —(CH₂)₂—, —(CH₂)₃— and —(CH₂)₄—.

3. Method according to claim 1, characterized in that the aliphatic alcohol is selected from methanol, ethanol, propanol, isopropanol and butanol, wherein methanol is pa icularly preferred.

4. Method according to claim 1, characterized in that the aliphatic alcohol in method step B) is used at a concentration of 5% by weight to 60% by weight, preferably from 10% by weight to 40% by weight, particularly preferably from 12% by weight to 30% by weight, wherein the percentages by weight relate to the total reaction batch.

5. Method according to claim 1, characterized in that method step B) is carried out in an aqueous environment.

6. Method according to claim 1, characterized in that method step B) is carried out in a temperature range from 5° C. to 80° C., preferably from 15° C. to 60° C., particularly preferably from 25° C. to 40° C.

7. Method according to claim 1, characterized in that method step B) is carried out in a pH range from 3 to 11, preferably from 5 to 9, particularly preferably from 6.5 to 8.

8. Method according to claim 1, characterized in that the enzyme E₁ is selected from the group
XP_003364701.1 (predicted equus caballus carboxylesterase isoform X2),
XP_005608328,1 (predicted equus caballus carboxylesterase isoform X3),
NP_388425.1 (Esterase 008 SD Bacillus subtilis),
Pig Liver Esterase 03 (commercially available from Enzymicals as ECS-PLE03),
Pig Liver Esterase 06 (commercially available from Enzymicals as ECS-PLE06), and
also CAO81735.1 (Alternative Pig liver esterase),
GI:576155 (Horse pancreatic lipase Chain A: 1HPLA),
G1:576156 (Horse pancreatic lipase Chain B: 1HPLB) and
AAC12774.1 (Esterase from Bacillus subtilis pdb 1.1KM brefeldin A esterase),
and also proteins having a polypeptide sequence in which up to 60% of the amino acid residues are modified compared with the abovementioned reference sequences by deletion, insertion, substitution or a combination thereof.

9. Method according to claim 1, characterized in that, in method step B), at least one enzyme selected from E₂, E₃ and E₄ is used,
wherein the enzyme E₂ catalyses the reaction of ω-hydroxycarboxylic esters to give the corresponding ω-carboxylic esters,
the enzyme E₃ catalyses the reaction of ω-oxocarboxylie esters to give the corresponding ω-aminocarboxylic esters and
the enzyme E₄ catalyses the reaction of ω-aminocarboxylic esters to give the corresponding the enzymes ω-aminocarboxylic acids.

10. Method according to claim 9, characterized in that, in method step B), an enzyme combination is used selected from E₁E₂, E₁E₃, E₁E₂, E₁E₂E₃, E₁E₃E₄, E₁E₂E₄ and E₁E₂E₃E₄.

11. Method according to claim 9, characterized in that E₂ is selected from alkanemonooxygenases, alcohol dehydrogenases and alcohol oxidases.

12. Method according to claim 9, characterized in that E₃ is selected from ω-transaminases.

13. Method according to claim 9, characterized in that E₄ is selected from the group
XP_003364701.1 (predicted equus eaballus carboxylesterase isoform X2),
XP_005608328.1 (predicted equus caballus carboxylesterase isoform X3),
NP_388425,1 (Esterase 008 SD Bacillus subtilis),
Pig Liver Esterase 03 (commercially available from Enzymicals as ECS-PLE03),
Pig Liver Esterase 06 (commercially available from Enzymicals as ECS-PLE06), and also CAO81735.1 (Alternative Pig liver esterase),
GI:576155 (Horse pancreatic lipase Chain A: 1HPL_{——}A),
GI:576156 (Horse pancreatic lipase Chain B: 1FIPL_{———}13) and
AAC12774.1 (Esterase from Bacillus subtilis pdb 1JKM brefeldin A esterase), and also proteins having a polypeptide sequence in which up to 60% of the amino acid residues are modified in comparison with the abovementioned reference sequences by deletion, insertion, substitution or a combination thereof.

14. Method according to claim 1, characterized in that the respective enzymes of the enzyme combinations selected from E₁, E₁E₂, E₁E₃, E₁E₄, E₁E₂E₃, E₁E₂E₃E₄, E₁E₂E₄ and E₁E₂E₃E₄ are used in the form of genetically modified cells which have an activity of the abovementioned enzymes which is increased in comparison with the wild type thereof.
